(19) United States
(12) Patent Application Publication
(10) Pub. No.: US 2025/0211822 A1
(43) Pub. Date: Jun. 26, 2025

(54) VIRTUAL AUDIO JACK FOR AUDIO PLAYBACK ON EXTERNAL DEVICE

(71) Applicant: SONY GROUP CORPORATION, TOKYO (JP)

(72) Inventors: PETER SHINTANI, SAN DIEGO, CA (US); BRANT CANDELORE, POWAY, CA (US); MAHYAR NEJAT, SAN DIEGO, CA (US)

(21) Appl. No.: 18/391,253

(22) Filed: Dec. 20, 2023

(51) Int. Cl. H04N 21/439; G06F 3/16

(52) U.S. Cl. CPC H04N 21/439; G06F 3/165; H04N 21/4307

(57) ABSTRACT

An electronic device and a method for virtual audio jack for audio playback on external device is disclosed. A first electronic device receives media content including audio content and video content. The first electronic device receives a user input indicative of a request to render the audio content on a second electronic device (for example, an external device) associated with the first electronic device. The first electronic device transmits a content request to a media server including the audio content, based on the received user input. The first electronic device renders the received media content on the first electronic device. The first electronic device controls the second electronic device to render the audio content, based on the transmitted content request. The rendering of the audio content on the second electronic device is based on a synchronization of the rendering of the media content on the first electronic device.

### FIELD

Various embodiments of the disclosure relate to media processing. More specifically, various embodiments of the disclosure relate to virtual audio jack for audio playback on external device.

### BACKGROUND

Advancements in the field of electrical and electronic technologies have led to development of various electronic devices such as, televisions, smartphones, tablets, and the like, that may render media content. The media content may include audio content and video content. An electronic device may include a display device and an audio device. The video content may be rendered on the display device and the audio content may be rendered on the audio device. The audio device may be a loudspeaker, a headphone, and the like. Certain electronic devices such as, televisions, may not include an audio jack. Thus, such electronic devices may not support connectivity to wired audio devices, such as, wired headsets, over the ear headphones, earphones, and the like. Therefore, in case the user is a hearing-impaired person, the user may not be able to effectively consume the audio content of the rendered media content. Further, a typical electronic device may support wireless connectivity to connect with a wireless headphone. However, in such cases, when the electronic device is wirelessly connected to the wireless headphone, internal speakers of the electronic device may be inactive. Therefore, in such cases, a person with normal hearing condition and a hearing-impaired person may not be able to consume the media content rendered on the electronic device simultaneously.

Limitations and disadvantages of conventional and traditional approaches will become apparent to one of skill in the art, through comparison of described systems with some aspects of the present disclosure, as set forth in the remainder of the present application and with reference to the drawings.

### SUMMARY

An electronic device and method for virtual audio jack for audio playback on external device is provided substantially as shown in, and/or described in connection with, at least one of the figures, as set forth more completely in the claims.

These and other features and advantages of the present disclosure may be appreciated from a review of the following detailed description of the present disclosure, along with the accompanying figures in which like reference numerals refer to like parts throughout.

### BRIEF DESCRIPTION OF THE DRAWINGS

FIG. 1 is a block diagram that illustrates an exemplary network environment for virtual audio jack for audio playback on external device, in accordance with an embodiment of the disclosure.

FIG. 2 is a block diagram that illustrates an exemplary first electronic device of FIG. 1, in accordance with an embodiment of the disclosure.

FIG. 3 is a block diagram that illustrates an exemplary second electronic device of FIG. 1, in accordance with an embodiment of the disclosure.

FIG. 4 is a diagram that illustrate an exemplary processing pipeline for the virtual audio jack for audio playback on external device, in accordance with an embodiment of the disclosure.

FIG. 5 is a diagram that illustrate an exemplary processing pipeline for rendering of an audio content based on the virtual audio jack, in accordance with an embodiment of the disclosure.

FIG. 6 is a diagram that illustrates an exemplary scenario for rendering of the audio content on external device based on a usage of the virtual audio jack, in accordance with an embodiment of the disclosure.

FIG. 7 is a flowchart that illustrates operations of an exemplary method for creation of the virtual audio jack for the audio playback on the external device, in accordance with an embodiment of the disclosure.

FIG. 8 is a flowchart that illustrates operations of an exemplary method for rendering of the audio content based on the virtual audio jack, in accordance with an embodiment of the disclosure.

### DETAILED DESCRIPTION

The following described implementation may be found in an electronic device and method for creation of the virtual audio jack for the audio playback on the external device. Exemplary aspects of the disclosure may provide an electronic device that may receive media content including audio content. The electronic device may receive a user input indicative of a request to render the audio content on a second electronic device associated with the first electronic device. Herein, a content request may be transmitted to a media server including the audio content, based on the received user input. The electronic device may render the received media content on the first electronic device. The electronic device may control the second electronic device to render the audio content, based on the transmitted content request. Herein, the rendering of the audio content on the second electronic device may be based on a synchronization of the rendering of the received media content on the first electronic device.

It may be appreciated certain electronic devices such as, televisions may not include an audio jack. Thus, such electronic devices may not support wired headsets, over the ear headphones, earphones, and the like. Such electronic devices include a display device and an audio device such that the video content may be rendered on the display device and the audio content may be rendered on the audio device. The audio device may be an internal speaker, an external speaker, and the like. However, as such electronic devices may not support audio devices, such as, wired headsets, over the ear headphones, earphones, and the like, therefore, in case the user is a hearing-impaired person, the user may not be able to effectively consume the audio content of the rendered media content. Further, a typical electronic device may support wireless connectivity to connect with a wireless headphone. However, in such cases, when the electronic device is wirelessly connected to the wireless headphone, internal speakers of the electronic device may be inactive. Therefore, in such cases, a person with normal hearing condition and a hearing-impaired person may not be able to consume the media content rendered on the electronic device simultaneously.

In order to address the aforesaid issues, the disclosed first electronic device and method may be used for creation virtual audio jack for audio playback on external device. The disclosed first electronic device may receive the media content including the audio content and the video content. In case a user wishes to use a headphone device, the user may provide the user input indicative of the request to render the audio content on the second electronic device associated with the first electronic device. Herein, the second electronic device may be a device such as, a smartphone that may include the audio jack. Upon reception of the user input, the content request may be transmitted to the media server including the audio content. Thereafter, the received media content may be rendered on the first electronic device. Herein, the video content may be rendered on a display device associated with the first electronic device and the audio content may be rendered on an audio device, such as, an internal speaker associated with the first electronic device. Moreover, the second electronic device may be controlled to render the audio content, based on the transmitted content request and the rendering of the received media content. Thus, a person who is associated with normal hearing condition may consume the received media content rendered on the first electronic device. Further, another person may use a headphone connected to a jack of the second electronic device during consumption of the audio content. Thus, the virtual audio jack may be created on the first electronic device.

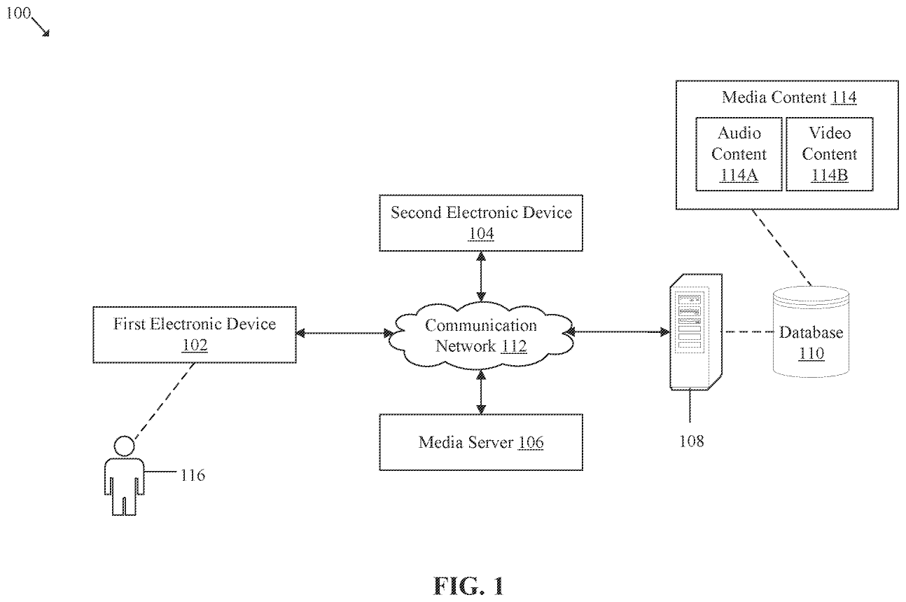

FIG. 1 is a block diagram that illustrates an exemplary network environment for virtual audio jack for audio playback on external device, in accordance with an embodiment of the disclosure. With reference to FIG. 1, there is shown a network environment 100. The network environment 100 may include a first electronic device 102, a second electronic device 104, a media server 106, a server 108, a database 110, and a communication network 112. The first electronic device 102 may communicate with the server 108, the media server 106, and/or the second electronic device 104 through one or more networks (such as, the communication network 112). The database 110 may include media content 114. The media content 114 may include audio content 114A and video content 114B. There is further shown, in FIG. 1, a user 116 that may be associated with the first electronic device 102.

The first electronic device 102 may include suitable logic, circuitry, interfaces, and/or code that may be configured to receive the media content 114 including the audio content 114A In some embodiments, the media content 114 may include the video content 114B. The first electronic device 102 may receive a user input indicative of a request to render the audio content 114A on the second electronic device 104 associated with the first electronic device 102. Herein, a content request may be transmitted to the media server 106 including the audio content 114A, based on the received user input. The first electronic device 102 may render the received media content 114 on the first electronic device 102. The first electronic device 102 may control the second electronic device 104 to render the audio content 114A, based on the transmitted content request, wherein the rendering of the audio content 114A on the second electronic device 104 may be based on a synchronization of the rendering of the received media content 114 on the first electronic device 102.

Examples of the first electronic device 102 may include, but are not limited to, a computing device, a smartphone, a cellular phone, a mobile phone, a gaming device, a mainframe machine, a server, a computer workstation, a device associated with a server farm (enabled with or hosting, for example, a computing resource, a memory resource, and a networking resource), and/or a consumer electronic (CE) device. The second electronic device 104 may include suitable logic, circuitry, interfaces, and/or code that may be configured to receive a user input indicative of a request to render the audio content 114A associated with media content 114 on the second electronic device 104 associated with the first electronic device 102. The content request may be transmitted to the media server 106 including the audio content 114A, based on the received user input. The second electronic device 104 may control the first electronic device 102 to render the media content 114 on the first electronic device 102. The second electronic device 104 may render the audio content 114A, based on the transmitted content request and the control of the rendering of the media content 114 on the first electronic device 102, wherein the rendering of the audio content 114A on the second electronic device 104 may be based on a synchronization of the rendering of the media content 114 on the first electronic device 102.

Examples of the second electronic device 104 may include, but are not limited to, a computing device, a smartphone, a cellular phone, a mobile phone, a gaming device, a mainframe machine, a server, a computer workstation, a device associated with a server farm (enabled with or hosting, for example, a computing resource, a memory resource, and a networking resource), and/or a consumer electronic (CE) device.

The media server 106 may include suitable logic, circuitry, and interfaces, and/or code that may be configured to receive the content request including the audio content 114A, based on the received user input. The media server 106 may be associated with a uniform resource identifier (URI). The media server 106 may be implemented as a cloud server and may execute operations through web applications, cloud applications, HTTP requests, repository operations, file transfer, and the like. Other example implementations of the media server 106 may include, but are not limited to, a database server, a file server, a web server, a media server, an application server, a mainframe server, a machine learning server (enabled with or hosting, for example, a computing resource, a memory resource, and a networking resource), or a cloud computing server.

The server 108 may include suitable logic, circuitry, and interfaces, and/or code that may be configured to receive the media content 114 including the audio content 114A and the video content 114B. The server 108 may receive the user input indicative of the request to render the audio content 114A on the second electronic device 104 associated with the first electronic device 102. Herein, the content request may be transmitted to the media server 106 including the audio content 114A, based on the received user input. The server 108 may render the received media content 114 on the first electronic device 102. The server 108 may control the second electronic device 104 to render the audio content 114A, based on the transmitted content request, wherein the rendering of the audio content on the second electronic device may be based on the synchronization of the rendering of the media content 114 on the first electronic device 102.

The server 108 may be implemented as a cloud server and may execute operations through web applications, cloud applications, HTTP requests, repository operations, file transfer, and the like. Other example implementations of the server 108 may include, but are not limited to, a database server, a file server, a web server, a media server, an application server, a mainframe server, a device associated with a server farm (enabled with or hosting, for example, a computing resource, a memory resource, and a networking resource), or a cloud computing server.

In at least one embodiment, the server 108 may be implemented as a plurality of distributed cloud-based resources by use of several technologies that are well known to those ordinarily skilled in the art. A person with ordinary skill in the art will understand that the scope of the disclosure may not be limited to the implementation of the server 108 and the first electronic device 102, as two separate entities. In certain embodiments, the functionalities of the server 108 can be incorporated in its entirety or at least partially in the first electronic device 102 without a departure from the scope of the disclosure. In certain embodiments, the server 108 may host the database 110. Alternatively, the server 108 may be separate from the database 110 and may be communicatively coupled to the database 110.

The database 110 may include suitable logic, interfaces, and/or code that may be configured to store the media content 114. The database 110 may be derived from data off a relational or non-relational database, or a set of comma-separated values (csv) files in conventional or big-data storage. The database 110 may be stored or cached on a device, such as, a server (e.g., the server 108) or the first electronic device 102. The device storing the database 110 may be configured to receive a query for the media content 114 from the first electronic device 102. In response, the device of the database 110 may be configured to retrieve and provide the queried media content 114 to the first electronic device 102, based on the received query.

In some embodiments, the database 110 may be hosted on a plurality of servers stored at the same or different locations. The operations of the database 110 may be executed using hardware including a processor, a microprocessor (e.g., to perform or control performance of one or more operations), a field-programmable gate array (FPGA), or an application-specific integrated circuit (ASIC). In some other instances, the database 110 may be implemented using software.

The communication network 112 may include a communication medium through which the first electronic device 102, the second electronic device 104, the media server 106, and/or the server 108 may communicate with one another. The communication network 112 may be one of a wired connection or a wireless connection. Examples of the communication network 112 may include, but are not limited to, the Internet, a cloud network, Cellular or Wireless Mobile Network (such as Long-Term Evolution and 5th Generation (5G) New Radio (NR)), satellite communication system (using, for example, a network of low earth orbit satellites), a Wireless Fidelity (Wi-Fi) network, a Personal Area Network (PAN), a Local Area Network (LAN), or a Metropolitan Area Network (MAN). Various devices in the network environment 100 may be configured to connect to the communication network 112 in accordance with various wired and wireless communication protocols. Examples of such wired and wireless communication protocols may include, but are not limited to, at least one of a Transmission Control Protocol and Internet Protocol (TCP/IP), User Datagram Protocol (UDP), Hypertext Transfer Protocol (HTTP), File Transfer Protocol (FTP), Zig Bee, EDGE, IEEE 802.11, light fidelity (Li-Fi), 802.16, IEEE 802.11s, IEEE 802.11g, multi-hop communication, wireless access point (AP), device to device communication, cellular communication protocols, and Bluetooth (BT) communication protocols.

The media content 114 may be a live media content, a pre-recorded media content, an over-the-air (OTA) media content, and the like. The media content 114 may include the audio content 114A. In some embodiments, the media content 114 may further include and the video content 114B. The audio content 114A may be an audio signal. The video content 114B may include a plurality of images that may be played sequentially over a time duration.

In operation, the first electronic device 102 may be configured to receive the media content 114 including the audio content 114A. The media content 114 may be the live media content, the pre-recorded media content, the over-the-air (OTA) media content, and the like. In an embodiment, the first electronic device 102 may not include an audio jack. In another embodiment, the first electronic device 102 may support wireless connectivity to connect with a wireless headphone. However, when the wireless connectivity to the wireless headphone is enabled, internal speakers of the first electronic device 102 may be inactive.

The first electronic device 102 may be configured to receive the user input indicative of the request to render the audio content 114A on the second electronic device 104 associated with the first electronic device 102. The content request may be transmitted to the media server 106 including the audio content 114A, based on the received user input. The second electronic device 104 may be a mobile device that may include a speaker and an audio jack. Thus, the second electronic device 104 may support headphones. Therefore, in case the user 116 associated with the second electronic device 104 wishes to use the headphone to listen to the audio content 114A, the user 116 may provide the user input indicative of the request to render the audio content 114A on the second electronic device 104.

The first electronic device 102 may render the received media content 114 on the first electronic device 102. The first electronic device 102 may include an audio device and a display device. Herein, the audio content 114A may be rendered on the audio device and the video content 114B may be rendered on the display device.

The first electronic device 102 may control the second electronic device 104 to render the audio content 114A, based on the transmitted content request, wherein the rendering of the audio content 114A on the second electronic device 104 may be based on the synchronization of the rendering of the received media content 114 on the first electronic device 102. The audio content 114A may be rendered on the second electronic device 104. As, the second electronic device 104 may support a headphone, and the headphone may be connected to an audio jack of the second electronic device 104. The user 116 associated with the second electronic device 104 may listen to the audio content 114A, via the headphone. Thus, a virtual audio jack may be created on the first electronic device 102.

In an embodiment, the second electronic device 104 may receive the user input indicative of the request to render the audio content 114A associated with the media content 114 on the second electronic device 104 associated with the first electronic device 102. Herein, the content request may be transmitted to the media server 106 including the audio content 114A, based on the received user input. Herein, the second electronic device 104 may support the headphone. However, the first electronic device 102 may not support the headphone. Therefore, in case a user, such as, the user 116, wishes to use the headphone, the user input indicative of the request to render the audio content 114A on the second electronic device 104 may be received.

The second electronic device 104 may control the first electronic device 102 to render the media content 114 on the first electronic device 102. Herein, the audio content 114A may be rendered on the audio device. The video content 114B may be rendered on the display device associated with the first electronic device 102.

The second electronic device 104 may render the audio content 114A, based on the transmitted content request, wherein the rendering of the audio content 114A on the second electronic device 102 may be based on a synchronization of the rendering of the received media content 114 on the first electronic device 102. The audio content 114A may be rendered on the second electronic device 104. As the second electronic device 104 may support the headphone, the user 116 associated with the second electronic device 104 may use the headphone to listen to the audio content 114A. Therefore, a virtual audio jack may be created on the first electronic device 102.

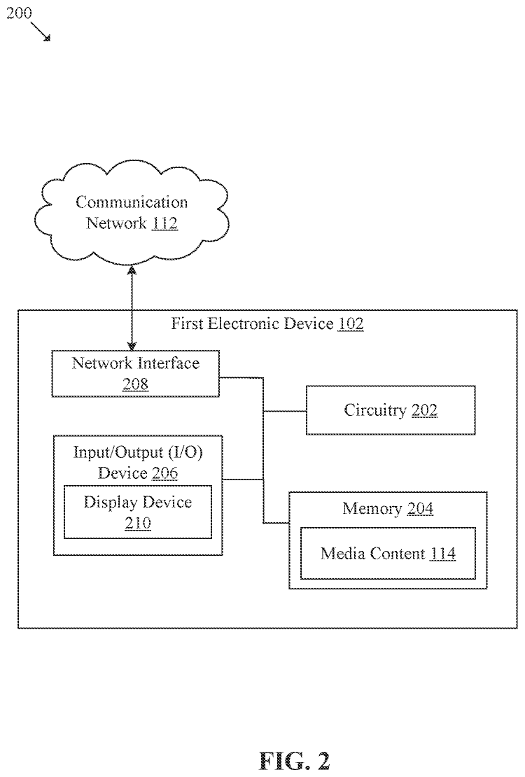

FIG. 2 is a block diagram that illustrates an exemplary first electronic device of FIG. 1, in accordance with an embodiment of the disclosure. FIG. 2 is explained in conjunction with elements from FIG. 1. With reference to FIG. 2, there is shown the exemplary first electronic device 102. The first electronic device 102 may include circuitry 202, a memory 204, an input/output (I/O) device 206, and a network interface 208. The memory 204 may include the media content 114. The input/output (I/O) device 206 may include a display device 210.

The circuitry 202 may include suitable logic, circuitry, and/or interfaces that may be configured to execute program instructions associated with different operations to be executed by the first electronic device 102. The operations may include media content reception, user input reception, headphone signal determination, headphone signal transmission, media content rendering, and audio content rendering. The circuitry 202 may include one or more processing units, which may be implemented as a separate processor. In an embodiment, the one or more processing units may be implemented as an integrated processor or a cluster of processors that perform the functions of the one or more specialized processing units, collectively. The circuitry 202 may be implemented based on a number of processor technologies known in the art. Examples of implementations of the circuitry 202 may be an X86-based processor, a Graphics Processing Unit (GPU), a Reduced Instruction Set Computing (RISC) processor, an Application-Specific Integrated Circuit (ASIC) processor, a Complex Instruction Set Computing (CISC) processor, a microcontroller, a central processing unit (CPU), and/or other control circuits.

The memory 204 may include suitable logic, circuitry, interfaces, and/or code that may be configured to store one or more instructions to be executed by the circuitry 202. The one or more instructions stored in the memory 204 may be configured to execute the different operations of the circuitry 202 (and/or the first electronic device 102). The memory 204 may be further configured to store the media content 114. Examples of implementation of the memory 204 may include, but are not limited to, Random Access Memory (RAM), Read Only Memory (ROM), Electrically Erasable Programmable Read-Only Memory (EEPROM), Hard Disk Drive (HDD), a Solid-State Drive (SSD), a CPU cache, and/or a Secure Digital (SD) card.

The I/O device 206 may include suitable logic, circuitry, interfaces, and/or code that may be configured to receive an input and provide an output based on the received input. For example, the I/O device 206 may receive a user input indicative of the request to render the audio content 114A on the second electronic device 104 associated with the first electronic device 102. The I/O device 206 may be further configured to display or render the media content 114. The I/O device 206 may include the display device 210. Examples of the I/O device 206 may include, but are not limited to, a display (e.g., a touch screen), a keyboard, a mouse, a joystick, a microphone, or a speaker. Examples of the I/O device 206 may further include braille I/O devices, such as, braille keyboards and braille readers.

The network interface 208 may include suitable logic, circuitry, interfaces, and/or code that may be configured to facilitate communication between the first electronic device 102, the second electronic device 104, the media server 106, and/or the server 108, via the communication network 112. The network interface 208 may be implemented by use of various known technologies to support wired or wireless communication of the first electronic device 102 with the communication network 112. The network interface 208 may include, but is not limited to, an antenna, a radio frequency (RF) transceiver, one or more amplifiers, a tuner, one or more oscillators, a digital signal processor, a coder-decoder (CODEC) chipset, a subscriber identity module (SIM) card, or a local buffer circuitry.

The network interface 208 may be configured to communicate via wireless communication with networks, such as the Internet, an Intranet, a wireless network, a cellular telephone network, a wireless local area network (LAN), or a metropolitan area network (MAN). The wireless communication may be configured to use one or more of a plurality of communication standards, protocols and technologies, such as Global System for Mobile Communications (GSM), Enhanced Data GSM Environment (EDGE), wideband code division multiple access (W-CDMA), Long Term Evolution (LTE), 5th Generation (5G) New Radio (NR), code division multiple access (CDMA), time division multiple access (TDMA), Bluetooth, Wireless Fidelity (Wi-Fi) (such as IEEE 802.11a, IEEE 802.11b, IEEE 802.11g or IEEE 802.11n), voice over Internet Protocol (VOIP), light fidelity (Li-Fi), Worldwide Interoperability for Microwave Access (Wi-MAX), a protocol for email, instant messaging, and a Short Message Service (SMS).

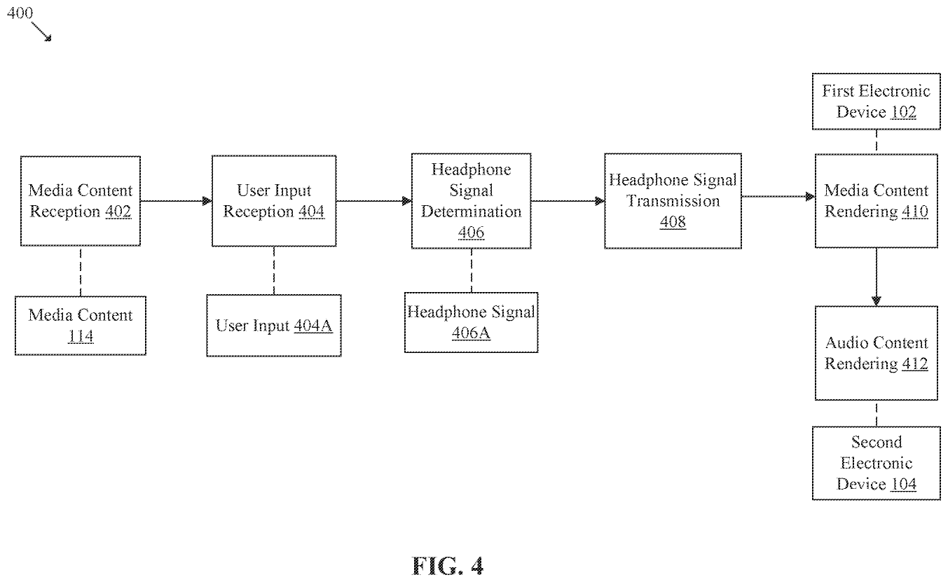

The display device 210 may include suitable logic, circuitry, and interfaces that may be configured to display or render the media content 114. The display device 210 may be a touch screen which may enable a user (e.g., the user 116) to provide a user-input via the display device 210. The touch screen may be at least one of a resistive touch screen, a capacitive touch screen, or a thermal touch screen. The display device 210 may be realized through several known technologies such as, but not limited to, at least one of a Liquid Crystal Display (LCD) display, a Light Emitting Diode (LED) display, a plasma display, or an Organic LED (OLED) display technology, or other display devices. In accordance with an embodiment, the display device 210 may refer to a display screen of a head mounted device (HMD), a smart-glass device, a see-through display, a projection-based display, an electro-chromic display, or a transparent display. Various operations of the circuitry 202 for determination of the virtual audio jack for audio playback on external device are described further, for example, in FIG. 4.

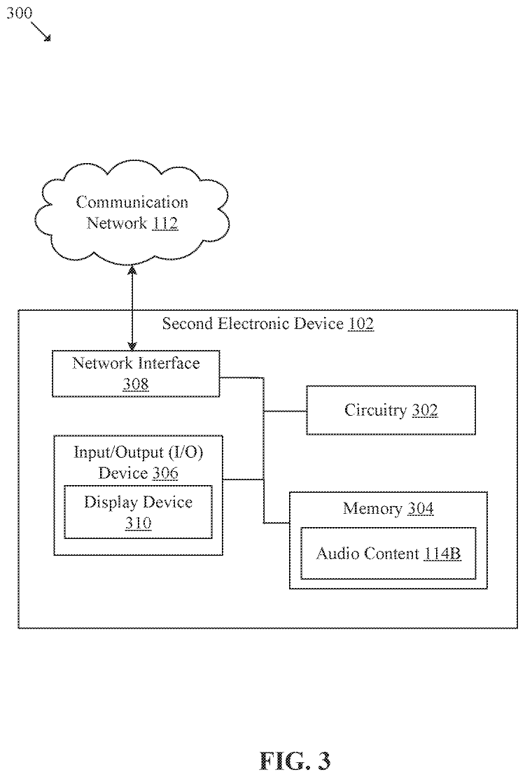

FIG. 3 is a block diagram that illustrates an exemplary second electronic device of FIG. 1, in accordance with an embodiment of the disclosure. FIG. 3 is explained in conjunction with elements from FIG. 1 and FIG. 2. With reference to FIG. 3, there is shown the exemplary second electronic device 104. The second electronic device 104 may include circuitry 302, a memory 304, an input/output (I/O) device 306, and a network interface 308. The memory 304 may include the audio content 114A. The input/output (I/O) device 306 may include a display device 310.

The circuitry 302 may include suitable logic, circuitry, and/or interfaces that may be configured to execute program instructions associated with different operations to be executed by the second electronic device 104. The operations may include user input reception, media content rendering, and audio content rendering. The circuitry 302 may include one or more processing units, which may be implemented as a separate processor. In an embodiment, the one or more processing units may be implemented as an integrated processor or a cluster of processors that perform the functions of the one or more specialized processing units, collectively. The circuitry 302 may be implemented based on a number of processor technologies known in the art. Examples of implementations of the circuitry 302 may be an X86-based processor, a Graphics Processing Unit (GPU), a Reduced Instruction Set Computing (RISC) processor, an Application-Specific Integrated Circuit (ASIC) processor, a Complex Instruction Set Computing (CISC) processor, a microcontroller, a central processing unit (CPU), and/or other control circuits.

The memory 304 may include suitable logic, circuitry, interfaces, and/or code that may be configured to store one or more instructions to be executed by the circuitry 302. The one or more instructions stored in the memory 304 may be configured to execute the different operations of the circuitry 302 (and/or the second electronic device 104). The memory 304 may be further configured to store the audio content 114A. Examples of implementation of the memory 304 may include, but are not limited to, Random Access Memory (RAM), Read Only Memory (ROM), Electrically Erasable Programmable Read-Only Memory (EEPROM), Hard Disk Drive (HDD), a Solid-State Drive (SSD), a CPU cache, and/or a Secure Digital (SD) card.

The I/O device 306 may include suitable logic, circuitry, interfaces, and/or code that may be configured to receive an input and provide an output based on the received input. For example, the I/O device 306 may receive a user input indicative of the request to render the audio content 114A on the second electronic device 104. The I/O device 306 may be further configured to render the audio content 114A. The I/O device 306 may include the display device 310. Examples of the I/O device 306 may include, but are not limited to, a display (e.g., a touch screen), a keyboard, a mouse, a joystick, a microphone, or a speaker. Examples of the I/O device 306 may further include braille I/O devices, such as, braille keyboards and braille readers.

The network interface 308 may include suitable logic, circuitry, interfaces, and/or code that may be configured to facilitate communication between the first electronic device 102, the second electronic device 104, the media server 106, and/or the server 108, via the communication network 112. The network interface 308 may be implemented by use of various known technologies to support wired or wireless communication of the second electronic device 104 with the communication network 112. The network interface 308 may include, but is not limited to, an antenna, a radio frequency (RF) transceiver, one or more amplifiers, a tuner, one or more oscillators, a digital signal processor, a coder-decoder (CODEC) chipset, a subscriber identity module (SIM) card, or a local buffer circuitry.

The network interface 308 may be configured to communicate via wireless communication with networks, such as the Internet, an Intranet, a wireless network, a cellular telephone network, a wireless local area network (LAN), or a metropolitan area network (MAN). The wireless communication may be configured to use one or more of a plurality of communication standards, protocols and technologies, such as Global System for Mobile Communications (GSM), Enhanced Data GSM Environment (EDGE), wideband code division multiple access (W-CDMA), Long Term Evolution (LTE), 5th Generation (5G) New Radio (NR), code division multiple access (CDMA), time division multiple access (TDMA), Bluetooth, Wireless Fidelity (Wi-Fi) (such as IEEE 802.11a, IEEE 802.11b, IEEE 802.11g or IEEE 802.11n), voice over Internet Protocol (VOIP), light fidelity (Li-Fi), Worldwide Interoperability for Microwave Access (Wi-MAX), a protocol for email, instant messaging, and a Short Message Service (SMS).

The display device 310 may include suitable logic, circuitry, and interfaces that may be configured to display or render the media content 114. The display device 310 may be a touch screen which may enable a user (e.g., the user 116) to provide a user-input via the display device 310. The touch screen may be at least one of a resistive touch screen, a capacitive touch screen, or a thermal touch screen. The display device 310 may be realized through several known technologies such as, but not limited to, at least one of a Liquid Crystal Display (LCD) display, a Light Emitting Diode (LED) display, a plasma display, or an Organic LED (OLED) display technology, or other display devices. In accordance with an embodiment, the display device 310 may refer to a display screen of a head mounted device (HMD), a smart-glass device, a see-through display, a projection-based display, an electro-chromic display, or a transparent display. Various operations of the circuitry 302 for rendering of an audio content based on the virtual audio jack are described further, for example, in FIG. 4.

FIG. 4 is a diagram that illustrate an exemplary processing pipeline for the virtual audio jack for audio playback on external device, in accordance with an embodiment of the disclosure. FIG. 4 is explained in conjunction with elements from FIG. 1, FIG. 2, and FIG. 3. With reference to FIG. 4, there is shown, an exemplary processing pipeline 400 that illustrates exemplary operations from 402 to 412 for the virtual audio jack for audio playback on external device. The exemplary operations 402 to 412 may be executed by any computing system, for example, by the first electronic device 102 of FIG. 1 or by the circuitry 202 of FIG. 2. FIG. 4 further includes the media content 114, a user input 404A, a headphone signal 406A, the first electronic device 102, and the second electronic device 104.

At 402, an operation of media content reception may be executed. The circuitry 202 may be configured to receive the media content 114 including the audio content 114A. In some embodiments, the media content 114 may include the video content 114B. The media content 114 may be a live media content, a pre-recorded media content, an over-the-air (OTA) media content, and the like. The video content 114B associated with the media content 114 may include a plurality of images that may be played sequentially over a period of time. The audio content 114A may include audio data associated with dialogues, background sound, music, and the like, associated with the media content 114. In an embodiment, the media content 114 may be stored in the database 110. Herein, the first electronic device 102 may request the database 110 for the media content 114. The database 110 may provide the media content 114 to the first electronic device 102 based on a validation of the request.

At 404, an operation of user input reception may be executed. The circuitry 202 may be configured to receive the user input 404A indicative of the request to render the audio content 114A on the second electronic device 104 associated with the first electronic device 102, wherein the content request may be transmitted to the media server 106 including the audio content 114A, based on the received user input 404A.

The second electronic device 104 may be associated with a user such as, the user 116. In an embodiment, the second electronic device 104 may be at least one of a mobile device or a media playback device. In an embodiment, the display device 310 may display a user interface (UI). The user input 404A may be received based on an interaction of a user such as, the user 116, with a UI element associated with the UI displayed on the display device 310. For example, the UI may include a first UI element that may include a question “Do you want to render the audio content 114A here?”. Further, the UI may include a second UI element and a third UI element that may correspond to a “yes” answer and a “no” answer associated with question. The user input 404A may be received based on a selection of the second UI element or the third UI element. Upon reception of the user input 404A, the content request may be transmitted to the media server 106. It may be appreciated that each electronic device may be associated with an identification number (such as, a model number, a unique media access control address, or a physical identifier of the electronic device) that may be specific to the corresponding electronic device. Thus, in order to let the media server 106, identify the second electronic device 104, the content request transmitted to the media server 106 may include the identification associated with the second electronic device 104.

At 406, an operation of headphone signal determination may be executed. In an embodiment, the circuitry 202 may be further configured to determine the headphone signal 406A associated with the audio content 114A. It may be appreciated that the headphone signal 406A may be a signal that may drive a headphone associated with the second electronic device 104. In an embodiment, the headphone signal 406A may be an electrical signal that may be provided to the headphone via a headphone port associated with the second electronic device 104. In another embodiment, the headphone signal 406A may be an amplified line level signal. The circuitry 202 may convert the audio content 114A into the headphone signal 406A.

In an embodiment, the circuitry 202 may be further configured to control an execution of a hearing test of a user such as, the user 116, on the second electronic device 104. In an example, the hearing test may include a plurality of beep tones and background noise that may be rendered on the second electronic device 104. A headphone may be connected to an audio jack of the second electronic device 104. The user 116 associated with the second electronic device 104 may wear the headphone and provide response based on the plurality of beep tones and background noise rendered on the second electronic device 104. Based on the provided response a hearing impairment information may be received or determined by the second electronic device 104.

The circuitry 202 may be configured to receive the hearing impairment information based on the execution of the hearing test. In an example, the hearing impairment information may indicate whether the user 116 associated with the second electronic device 104 is able to hear a first beep tone, a second beep tone, a background noise, and a third beep tone.

The circuitry 202 may be configured to determine a hearing profile of the user 116 associated with the second electronic device 104 based on the received hearing impairment information, wherein the headphone signal 406A may be determined further based on the determined hearing profile. The received hearing impairment information may be used to determine the hearing profile of the user 116.

In an embodiment, the determined hearing profile may include at least one of a hearing capability of the user 116, an audio characteristic associated with the user 116, a preferred bass of the user 116, a preferred treble of the user 116, or a preferred amplitude of the user 116. The hearing capability of the user 116 may indicate whether the user 116 faces issues in hearing. In an example, the hearing capability may be “normal” in case the user 116 associated with the second electronic device 104 may be able to hear sounds in a range of “10” decibels to “15” decibels. The hearing capability may be “mild hearing loss” in case the user 116 associated with the second electronic device 104 may be able to hear sounds above “30” decibels. The hearing capability may be “moderate hearing loss” in case the user 116 associated with the second electronic device 104 may be able to hear sounds above “41” decibels. The hearing capability may be “moderately severe hearing loss” in case the user 116 associated with the second electronic device 104 may be able to hear sounds above “56” decibels. The hearing capability may be “severe hearing loss” in case the user 116 associated with the second electronic device 104 may be able to hear sounds above “71” decibels. The audio characteristic associated with the user 116 may be a pitch level, an intensity, and a quality of an audio content such as, the audio content 114A preferred by the user 116. The preferred bass of the user 116 may be tones of low frequency or low pitch preferred by the user 116. In an example, the preferred bass of the user 116 may be “fender jazz bass”. The preferred treble of the user 116 may be tones of high frequency or high pitch preferred by the user 116. The preferred amplitude of the user 116 may be an intensity level preferred by the user 116.

Based on the determined hearing profile, the headphone signal 406A may be determined. For example, the determined hearing profile may indicate that the user 116 may have “mild hearing loss” and may prefer “fender jazz bass”. Further, the determined hearing profile may indicate that the preferred amplitude of the user 116 may be “32” decibels. The headphone signal 406A may be determined such that the bass of the headphone signal 406A may be “fender jazz bass” and the amplitude of the headphone signal 406A may be “32” decibels.

In an embodiment, the circuitry 202 may be further configured to receive a hearing profile of a user such as, the user 116, associated with the second electronic device 104, wherein the headphone signal 406A may be determined further based on the received hearing profile. Herein, instead of the execution of the hearing test of the user 116 on the second electronic device 104, the hearing profile of the user 116 may be directly received. In an example, a user interface may be rendered on the display device 310 of the second electronic device 104. The user interface may include a first UI element, a second UI element, and a third UI element. The first UI element may include a statement “Please provide your hearing capability”. The second UI element may include a statement “Please provide the preferred”. The third UI element may include a statement “Please provide the preferred treble of the user 116. User responses may be received through the first UI element, the second UI element, and the third UI element. Based on the received user responses, the hearing profile of the user 116 may be received. Based on the received hearing profile, the headphone signal 406A may be determined.

In an embodiment, the circuitry 202 may be further configured to apply an audio equalization on the audio content 114A based on the received hearing profile, wherein the headphone signal 406A may be determined further based on the application of the audio equalization. The audio equalization may be a process of adjusting loudness or intensity of one of one or more frequency bands present in the audio content 114A. In an example, the hearing profile of the user 116 associated with the second electronic device 104 may indicate that the user may have the “moderate hearing loss”. Based on the hearing profile, the audio equalization may be applied on the audio content 114A to determine the headphone signal 406A by enhancing the loudness of certain frequency ranges present in the audio content 114A to “41” decibels. In another example, the hearing profile of the user 116 associated with the second electronic device 104 may indicate that the user may have the “moderate hearing loss” in a left ear and the “mild hearing loss” in a right ear. Based on the hearing profile, the audio equalization may be applied on the audio content 114A to determine the headphone signal 406A such that an audio output for the left ear may be louder than the audio output for the right ear.

In an embodiment, the circuitry 202 may be further configured to apply a dialogue enhancement on the audio content 114A based on the received hearing profile, wherein the headphone signal 406A may be determined further based on the application of the dialogue enhancement. It may be appreciated that a dialogue enhancement may be process to increase an intensity of a speech portion of the audio content 114A. An intensity of the background content of the audio content 114A may be unchanged so that an overall intensity of the audio content 114A is unchanged. That is, the dialogue enhancement may suppress the background content of the audio content 114A so that the speech portion of the audio content 114A may be clearly heard by a person such as, the user 116. In an example, the hearing profile of the user 116 associated with the second electronic device 104 may indicate that the user 116 may have the “moderately severe hearing loss”. Herein, the dialogue enhancement may be applied on the audio content 114A so that a loudness of the speech portion of the audio content 114A may be increased so that the user 116 having the “moderately severe hearing loss” may be able to hear the speech portion of the audio content 114A clearly.

In an embodiment, the circuitry 202 may be further configured to receive a viewing profile of the user 116 associated with the second electronic device 104. The circuitry 202 may be further configured to determine an audio description associated with at least one portion of the received media content 114 based on the viewing profile. The circuitry 202 may be further configured to insert the determined audio description in the determined headphone signal 406A. The viewing profile may indicate a visibility condition of the user 116.

In an example, the viewing profile of the user 116 associated with the second electronic device 104 may indicate that the user 116 may have a “moderate visibility loss”. Herein, the user 116 associated with the second electronic device 104 may be unable to watch certain portions of the media content 114. Therefore, the user 116 may miss such portions of the media content 114. In order to mitigate aforesaid issues, the audio description of such media portions of the media content 114 may be determined. For example, the audio description may include a transcription of background content present in a media portion having a time stamp “01:01:00” to time stamp “01.08:09”. The audio description may be played to help the user 116 to grasp the media portion from the time stamp “01:01:00” to the time stamp “01.08:09”

At 408, an operation of headphone signal transmission may be executed. The circuitry 202 may be further configured to transmit the determined headphone signal 406A to the second electronic device 104, based on the transmission of the content request. Upon determination of the headphone signal 406A, the determined headphone signal 406A may be transmitted to the second electronic device 104, via the communication network 112. Herein, the media server 106 may signal that audio content 114A may correspond to a first audio stream and the determined headphone signal 406A may correspond to a second audio stream. Further, the media server 106 may signal the first electronic device 102 that a session associated with rendering of the first audio stream should be maintained.

At 410, an operation of received media content rendering may be executed. The circuitry 202 may be further configured to render the received media content 114 on the first electronic device 102. Herein, the audio content 114A may be played on an audio device associated with the first electronic device 102 and the video content 114B may be displayed on the display device 210.

At 412, an operation of audio content rendering may be executed. The circuitry 202 may be further configured to control the second electronic device 104 to render the audio content 114A, based on the transmitted content request. The rendering of the audio content 114A on the second electronic device 104 may be based on a synchronization of the rendering of the received media content 114 on the first electronic device 102. In an embodiment, the control of the rendering of the audio content 114A on the second electronic device 104 may be further based on the transmitted headphone signal 406A. In an example, a user such as, the user 116, associated with the second electronic device 104 may have hearing issues. Hence, the user 116 may wish to listen to the audio associated with the media content 114, via a headphone. The first electronic device 102 may not support the headphone. In such a case, the circuitry 202 may convert the audio content 114A into the headphone signal 406A that may be transmitted to the second electronic device 104. The second electronic device 104 may support a headphone. The headphone signal 406A may drive a headphone associated with the second electronic device 104.

It may be noted that the first audio stream and the second audio stream may be streamed equivalently. Herein, commands such as play, pause, rewind, fast forward, and the like may be controlled from the first electronic device 102 and/or the second electronic device 104. Herein, playback of the media content 114 may be at exactly a same location in the first audio stream and the second audio stream. Closing a session on any one device, such as the first electronic device 102 may lead to a display of a user interface (UI) that may ask the user 116 whether the rendering of the media content 114 on the first electronic device 102 may need to be stopped or whether the rendering of the audio content 114A on the second electronic device 102 may need to be stopped. Based on a response received, the session on the first electronic device 102 or the second electronic device 102 may be stopped.

Alternatively, in some cases, the first electronic device 102 may request the second electronic device 102 to transmit information associated with an Internet Protocol (IP) address of the second electronic device 102. Thereafter, based on the received IP address, the received IP address and an ID of the second electronic device 102 may be transmitted to the media server 106. Further, the first electronic device 102 may transmit the media content 114, a current play back location in the media content 114, and the like to the media server 106. Thereafter, the media server 106 may communicate with the second electronic device 104 based on the received IP address. Hereafter, the audio content 114A may be rendered on the second electronic device 102. Moreover, in an embodiment, the media server 106 may transmit the second media stream to the second electronic device 104.

In an embodiment, the circuitry 202 may be further configured to receive identification information associated with the second electronic device 104. The identification information may be an identification (ID) number associated with the second electronic device 104. Each electronic device may be associated with a unique ID number. Upon reception of the identification information associated with the second electronic device 104, the circuitry 202 may register the associated with the second electronic device 104 on to the first electronic device 102.

The circuitry 202 may be further configured to transmit the received identification information to the media server 106, wherein the control of the rendering of the audio content 114A on the second electronic device 104 may be further based on the transmitted identification information. The media server 106 may compare the identification information received from the circuitry 202 with the identification included in the control request. In case the identification information received from the circuitry 202 matches with the identification included in the control request then the circuitry 202 may control of the rendering of the audio content 114A on the second electronic device 104. In case the identification information received from the circuitry 202 does not match with the identification included in the control request, then the audio content 114A may not be rendered on the second electronic device 104.

In an embodiment, the circuitry 202 may be further configured to determine time information associated with the audio content 114A. The circuitry 202 may be further configured to synchronize the transmitted headphone signal 406A with the received media content 114 rendered on the first electronic device 102, wherein the control of the rendering of the audio content 114A on the second electronic device 104 may be further based on the synchronization of the transmitted headphone signal 406A. The determined time information associated with the audio content 114A may include a plurality of time stamps and audio information associated with each time stamp. For example, the determined time information may include a time stamp “00:00:00-00:00:20”, “00:00:20-00:01:10”, and “00:01:10-00:01:00”. Further, the determined time information may include the audio information as “background music” for the time stamp “00:00:00-00:00:20”, the audio information as “dialogue delivery of Mary” for the time stamp “00:00:20-00:01:10”, and the audio information as “action scene” for the time stamp “00:01:10-00:01:00”. Upon determination of the time information, the transmitted headphone signal 406A may be synchronized with the received media content 114 rendered on the first electronic device 102. Further, the audio content 114A may be rendered on the second electronic device 104 further based on the synchronization of the transmitted headphone signal 406A. That is, for example, in case a time stamp associated with the received media content 114 rendered on the first electronic device 102 is “00:10:01”, then the time stamp associated with the audio content 114A rendered on the second electronic device 104 may be also “00:10:01”. The synchronization of the transmitted headphone signal 406A may prevent a lag between the received media content 114 rendered on the first electronic device 102 and the audio content 114A may be rendered on the second electronic device 104. Thus, the synchronization of the transmitted headphone signal 406A may enrich a viewing and listening experience of the user such as, the user 116, associated with the second electronic device 104.

In an embodiment, the circuitry 202 may be further configured to determine whether the received media content 114 corresponds to over-the-air (OTA) content. The circuitry 202 may be further configured to determine a broadcaster associated with the received media content 114 based on the determination that the received media content 114 corresponds to the OTA content. The circuitry 202 may be further configured to receive a uniform resource identifier (URI) associated with the media server 106 based on the determined broadcaster. The circuitry 202 may be further configured to transmit the received URI associated with the media server 106 to the second electronic device 104, based on the received user input, wherein the control of the rendering of the audio content 114A on the second electronic device 104 may be further based on the transmitted URI associated with the media server 106.

It may be appreciated that the OTA content may be delivered via usage of a wireless communication system. Herein, an antenna associated with the first electronic device 102 may receive radio signals associated with the OTA content from the broadcaster. In an example, the broadcaster may be a television station. In case the received media content 114 corresponds to the OTA content, the URI associated with the media server 106 may be received. The URI may be a unique character sequence that may be associated with the media server 106. The URI may be used to identify the media server 106. The received URI may be transmitted to the second electronic device 104. The second electronic device 104 may then communicate with the media server 106 based on the received URI. The second electronic device 104 receive the audio content 114A from the media server 106. The audio content 114A received from the media server 106 may be rendered on the second electronic device 104.

The disclosed first electronic device 102 may thus create the virtual audio jack for audio playback. In case a user such as, the user 116, associated with the second electronic device 104 wishes to use a headphone device, the user 116 may provide the user input 404A indicative of the request to render the audio content 114A on the second electronic device 104 associated with the first electronic device 102. Herein, the second electronic device 104 may be a device such as, a smartphone, that may include an audio jack. Upon reception of the user input 404A, the content request may be transmitted to the media server 106 including the audio content 114A. Thereafter, the received media content 114 may be rendered on the first electronic device 102. Herein, the video content 114B may be rendered on the display device 210 associated with the first electronic device 102 and the audio content 114A may be rendered on an audio device such as, an internal speaker associated with the first electronic device 102. Moreover, the second electronic device 104 may be controlled to render the audio content 114A, based on the transmitted content request and the rendering of the received media content 114. Thus, a person who is associated with normal hearing conditions may consume the received media content 114 rendered on the first electronic device 102. Further, another person (for example, a hearing-impaired person) may use the headphone connected to an audio jack of the second electronic device 104 to consume the audio content 114A. Thus, the virtual audio jack may be created on the first electronic device 102. Further, in some embodiments, the audio content 114A rendered on the second electronic device 104 may be synchronized with the media content 114 rendered on the first electronic device 102. The synchronization may ensure a rich viewing and listening experience to a user associated with the second electronic device 104. Usage of hearing profile and/or viewing profile for rendering of the audio content 114A on the second electronic device 104 may further enhance the media consumption experience of a user associated with the second electronic device 104

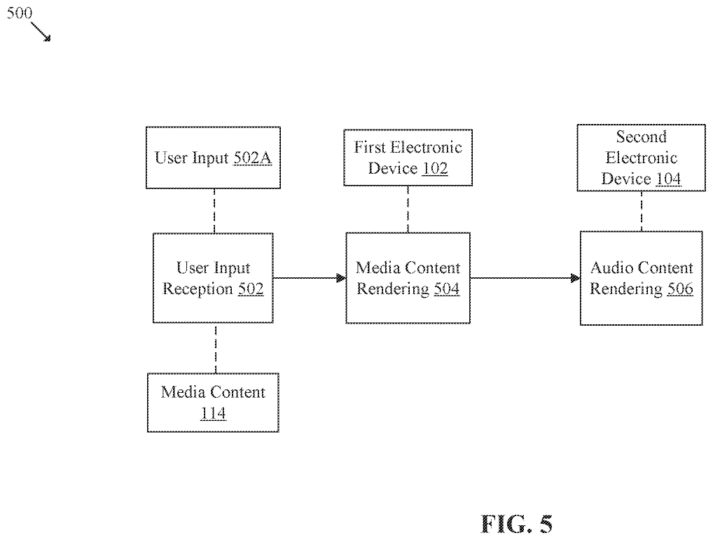

FIG. 5 is a diagram that illustrate an exemplary processing pipeline for rendering of an audio content based on the virtual audio jack, in accordance with an embodiment of the disclosure. FIG. 5 is explained in conjunction with elements from FIG. 1, FIG. 2, FIG. 3, and FIG. 4. With reference to FIG. 5, there is shown, an exemplary processing pipeline 500 that illustrates exemplary operations from 502 to 506 for the virtual audio jack for audio playback on external device. The exemplary operations 502 to 506 may be executed by any computing system, for example, by the second electronic device 104 of FIG. 1 or by the circuitry 302 of FIG. 3. FIG. 4 further includes the media content 114, a user input 502A, the first electronic device 102, and the second electronic device 104.

At 502, an operation of user input reception may be executed. The circuitry 302 may be configured to receive the user input 502A indicative of the request to render audio content 114A associated with the media content 114 on the second electronic device 104 associated with the first electronic device 102, wherein the content request may be transmitted to the media server 106 including the audio content 114A, based on the received user input 502A. In an example, a UI may be displayed on the display device 310. The user input 502A may be received through a UI element associated with the UI displayed on the display device 310. For example, a person such as, the user 116, associated with the second electronic device 104 may wish to use a headphone. The first electronic device 102 may not support the headphone. Hence, the user input 502A may be provided by the user 116 as a request to render audio content 114A associated with the media content 114 on the second electronic device 104. Details related to the user input reception are further provided, for example, in FIG. 4 (at 404).

At 504, an operation of media content rendering may be executed. The circuitry 302 may be configured to control the first electronic device 102 to render the media content 114 on the first electronic device 102. Herein, the media content 114 may be rendered on the first electronic device 102, such that the video content 114B is rendered on the display device 210 of the first electronic device 102 and the audio content 114A may be rendered on an audio device associated with the first electronic device 102. Details related to the rendering of the media content 114 on the first electronic device 102 are further provided, for example, in FIG. 4 (at 410).

At 506, an operation of audio content rendering may be executed. The circuitry 302 may be configured to render the audio content 114A, based on the transmitted content request and the control of the rendering of the media content 114 on the first electronic device 102. The rendering of the audio content 114A on the second electronic device 104 may be based on the synchronization of the rendering of the media content 114 on the first electronic device 102. Herein, the audio content 114A associated with the media content 114 may be rendered on an audio device associated with the second electronic device 104. For example, the audio content 114A may be rendered on the headphone connected to a jack of the second electronic device 104. Details related to the rendering of the audio content 114A on the second electronic device 104 are further provided, for example, in FIG. 4 (at 412).

The second electronic device 104 may thus create the virtual audio jack for audio playback on the first electronic device 102. In case a user such as, the user 116, associated with the second electronic device 104 wishes to use a headphone device, the user 116 may provide the user input 404A indicative of the request to render the audio content 114A on the second electronic device 104. Upon reception of the user input 404A, the content request may be transmitted to the media server 106 including the audio content 114A. Thereafter, the second electronic device 104 may control the first electronic device 102 to render the received media content 114. Moreover, the second electronic device 104 may render the audio content 114A, based on the transmitted content request and the rendering of the received media content 114. Thus, the person who is associated with normal hearing conditions may consume the received media content 114 rendered on the first electronic device 102. Further, another person such as, the user 116, may use the headphone connected to the audio jack of the second electronic device 104 to consume the audio content 114A. Thus, the first electronic device 102 may create the virtual audio jack on the first electronic device 102. Further, in some embodiments, the audio content 114A rendered on the second electronic device 104 may be synchronized with the media content 114 rendered on the first electronic device 102. The synchronization may ensure a rich viewing experience for the user 116 associated with the second electronic device 104. Usage of hearing profile and/or viewing profile for rendering of the audio content 114A on the second electronic device 104 may further enhance the viewing experience of the user 116 associated with the second electronic device 104

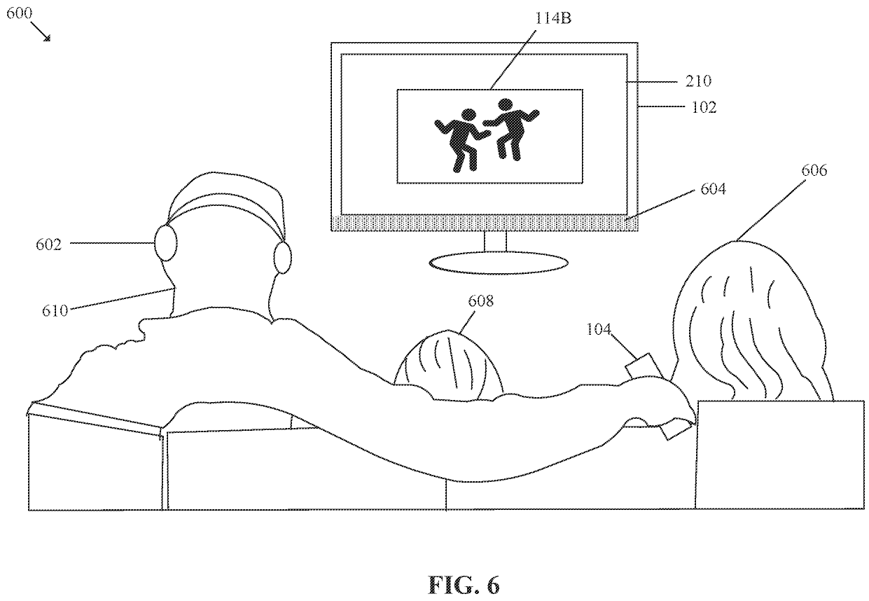

FIG. 6 is a diagram that illustrate an exemplary scenario for rendering of an audio content based on the virtual audio jack, in accordance with an embodiment of the disclosure. FIG. 6 is described in conjunction with elements from FIG. 1, FIG. 2, FIG. 3, FIG. 4, and FIG. 5. With reference to FIG. 6, there is shown an exemplary scenario 600. The scenario 600 may include the first electronic device 102, the second electronic device 104, and a headphone 602. The first electronic device 102 may include the display device 210, the video content 114B, and an audio device 604. There is further shown, in FIG. 1, a first user 606 and a second user 608 (both associated with the first electronic device 102) and a third user 610 (that may be associated with the second electronic device 104). A set of operations associated with the scenario 600 is described herein.

With reference to FIG. 6, the circuitry 202 may render the video content 114B on the display device 210 of the first electronic device 102. Further, the audio content 114A may be rendered on the first electronic device 102 via the audio device 604. The first electronic device 102 shown in FIG. 6 corresponds to a television. The first user 606 and the second user 608 may have normal hearing conditions. Therefore, the first user 606 and the second user 608 may consume the media content 114 based on the rendering of the media content 114 on the first electronic device 102. However, the third user 610 may be associated with a hearing impairment condition. For example, the hearing capability of the third user 610 may be the “moderate hearing loss”. Thus, the third user 610 may need to use the headphone 602 in order to consume the audio content 114A. The first electronic device 102 may not support the headphone 602. Thus, the third user 610 may place the request to render the audio content 114A on the second electronic device 104 based on the user input 404A. Thereafter, the first electronic device 102 may control the rendering of the audio content 114A on the second electronic device 104. The headphone 602 may be connected to the second electronic device 104. The third user 610 may view the video content 114B rendered on the display device 210 of the first electronic device 102 and may listen to the audio content 114A from the headphone 602.

It should be noted that scenario 600 of FIG. 6 is for exemplary purposes and should not be construed to limit the scope of the disclosure.

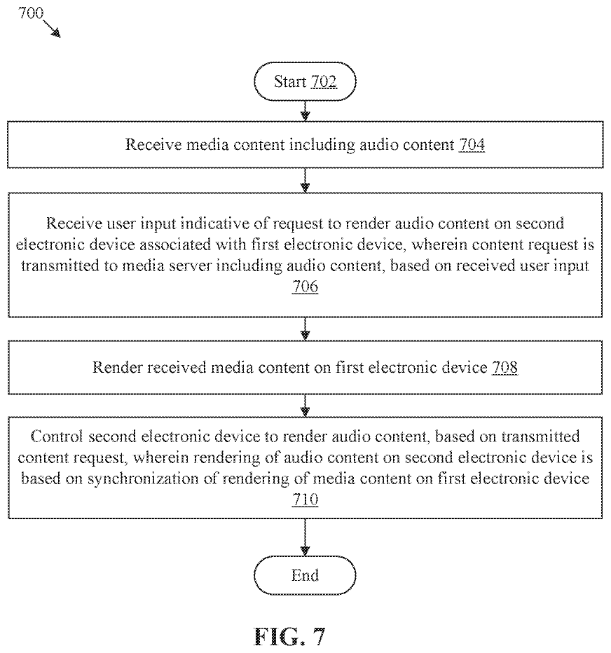

FIG. 7 is a flowchart that illustrates operations of an exemplary method for creation of the virtual audio jack for the audio playback on the external device, in accordance with an embodiment of the disclosure. FIG. 7 is described in conjunction with elements from FIG. 1, FIG. 2, FIG. 3, FIG. 4, FIG. 5, and FIG. 6. With reference to FIG. 7, there is shown a flowchart 700. The flowchart 700 may include operations from 702 to 710 and may be implemented by the first electronic device 102 of FIG. 1 or by the circuitry 202 of FIG. 2. The flowchart 700 may start at 702 and proceed to 704.

At 704, the media content 114 including the audio content 114A may be received. The circuitry 202 may be configured to receive the media content 114 including the audio content 114A. Details related to the reception of the media content 114 are further provided, for example, in FIG. 4 (at 402).

At 706, the user input indicative of the request to render the audio content 114A on the second electronic device 104 associated with the first electronic device 102 may be received. The content request may be transmitted to the media server 106 including the audio content 114A, based on the received user input 404A. The circuitry 202 may be configured to receive the user input indicative of the request to render the audio content 114A on the second electronic device 104 associated with the first electronic device 102, wherein the content request may be transmitted to the media server 106 including the audio content 114A, based on the received user input 404A. Details related to the reception of user input are further provided, for example, in FIG. 4 (at 404).

At 708, the received media content 114 may be rendered on the first electronic device 102. The circuitry 202 may be configured to render the received media content 114 on the first electronic device 102. Details related to the rendering of the media content 114 are further provided, for example, in FIG. 4 (at 410).

At 710, the second electronic device 104 may be controlled to render the audio content 114A, based on the transmitted content request. The rendering of the audio content 114A on the second electronic device 104 may be based on a synchronization of the rendering of the received media content 114 on the first electronic device 102. The circuitry 202 may be configured to control the second electronic device 104 to render the audio content 114A, based on the transmitted content request. The rendering of the audio content 114A on the second electronic device 104 may be based on a synchronization of the rendering of the received media content 114 on the first electronic device 102. Details related to the rendering of the audio content 114A are further provided, for example, in FIG. 4 (at 412). Control may pass to end.

Although the flowchart 700 is illustrated as discrete operations, such as, 704, 706, 708, and 710, the disclosure is not so limited. Accordingly, in certain embodiments, such discrete operations may be further divided into additional operations, combined into fewer operations, or eliminated, depending on the implementation without detracting from the essence of the disclosed embodiments.

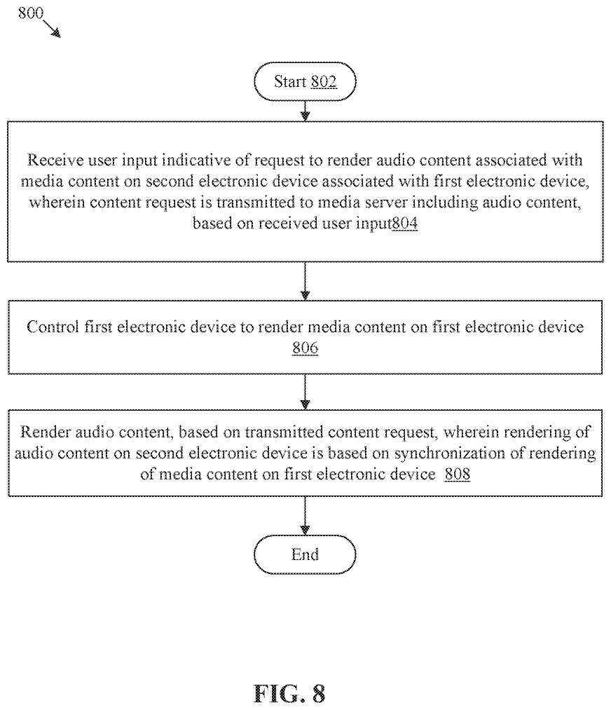

FIG. 8 is a flowchart that illustrates operations of an exemplary method for rendering of the audio content based on the virtual audio jack, in accordance with an embodiment of the disclosure. FIG. 8 is described in conjunction with elements from FIG. 1, FIG. 2, FIG. 3, FIG. 4, FIG. 5, FIG. 6, and FIG. 7. With reference to FIG. 8, there is shown a flowchart 800. The flowchart 800 may include operations from 802 to 808 and may be implemented by the second electronic device 104 of FIG. 1 or by the circuitry 302 of FIG. 3. The flowchart 800 may start at 802 and proceed to 804.

At 804, the user input 502A indicative of the request to render the audio content 114A associated with media content 114 may be received on the second electronic device 104 associated with the first electronic device 102, wherein the content request may be transmitted to the media server 106 including the audio content 114A, based on the received user input 502A. The circuitry 302 may be configured to receive the user input 502A indicative of the request to render the audio content 114A associated with media content 114 on the second electronic device 104 associated with the first electronic device 102, wherein the content request may be transmitted to the media server 106 including the audio content 114A, based on the received user input 502A. Details related to the reception of the media content 114 are further provided, for example, in FIG. 5 (at 502).

At 806, the first electronic device 102 may be controlled to render the media content 114 on the first electronic device 102. The circuitry 302 may be configured to control the first electronic device 102 to render the media content 114 on the first electronic device 102. Details related to the rendering of the media content 114 are further provided, for example, in FIG. 5 (at 504).

At 808, the audio content 114A may be rendered based on the transmitted content request. The rendering of the audio content 114 on the second electronic device 104 may be based on a synchronization of the rendering of the media content 114 on the first electronic device 102. The circuitry 302 may be configured to render the audio content 114A, based on the transmitted content request. Details related to the rendering of the audio content 114A are further provided, for example, in FIG. 5 (at 506). Control may pass to end.

Although the flowchart 800 is illustrated as discrete operations, such as, 804, 806, and 808, the disclosure is not so limited. Accordingly, in certain embodiments, such discrete operations may be further divided into additional operations, combined into fewer operations, or eliminated, depending on the implementation without detracting from the essence of the disclosed embodiments.

Various embodiments of the disclosure may provide a non-transitory computer-readable medium and/or storage medium having stored thereon, computer-executable instructions executable by a machine and/or a computer to operate a first electronic device (for example, the first electronic device 102 of FIG. 1). Such instructions may cause the first electronic device 102 to perform operations that may include reception of media content (e.g., the media content 114) including audio content (e.g., the audio content 114A). The operations may further include reception of a user input (e.g., the user input 404A) indicative of the request to render the audio content 114A on a second electronic device (e.g., the second electronic device 104) associated with the first electronic device 102, wherein the content request may be transmitted to a media server (e.g., the media server 106) including the audio content 114A, based on the received user input 404A. The operations may further include rendering the received media content 114 on the first electronic device 102. The operations may further include controlling the second electronic device 104 to render the audio content 114A, based on the transmitted content request. The rendering of the audio content 114A on the second electronic device 104 may be based on a synchronization of the rendering of the media content 114 on the first electronic device 102.

Various embodiments of the disclosure may provide a non-transitory computer-readable medium and/or storage medium having stored thereon, computer-executable instructions executable by a machine and/or a computer to operate a second electronic device (for example, the second electronic device 104 of FIG. 1). Such instructions may cause the reception of a user input (e.g., the user input 502A) indicative of the request to render the audio content 114A associated with the media content 114 on the second electronic device 104 associated with the first electronic device 102. The content request may be transmitted to the media server 106 including the audio content 114A, based on the received user input 502A. The operations may further include control of the first electronic device 102 to render the media content 114 on the first electronic device 102. The operations may further include render the audio content 114A, based on the transmitted content request. The rendering of the audio content 114A on the second electronic device 104 may be based on a synchronization of the rendering of the media content 114 on the first electronic device 102.

Exemplary aspects of the disclosure may provide a first electronic device (such as, the first electronic device 102 of FIG. 1) that includes circuitry (such as, the circuitry 202). The circuitry 202 may be configured to receive the media content 114 including the audio content 114A and the video content 114B. The circuitry 202 may be configured to receive the user input 404A indicative of the request to render the audio content 114A on the second electronic device 104 associated with the first electronic device 102, wherein the content request may be transmitted to the media server 106 including the audio content 114A, based on the received user input. The circuitry 202 may be configured to render the received media content 114 on the first electronic device 102. The circuitry 202 may be configured to control the second electronic device 104 to render the audio content 114A, based on the transmitted content request and the rendering of the received media content 114. The rendering of the audio content 114A on the second electronic device 104 may be based on the synchronization of the rendering of the media content 114 on the first electronic device 102.

Exemplary aspects of the disclosure may provide a second electronic device (such as, the second electronic device 104 of FIG. 1) that includes circuitry (such as, the circuitry 302). The circuitry 302 may be configured to receive the user input 502A indicative of the request to render the audio content 114A associated with the media content 114 on the second electronic device 104 associated with the first electronic device 102. The content request may be transmitted to the media server 106 including the audio content 114A, based on the received user input 502A. The circuitry 302 may be configured to control the first electronic device 102 to render the media content 114 on the first electronic device 102. The circuitry 302 may be configured to render the audio content 114A, based on the transmitted content request. The rendering of the audio content 114A on the second electronic device 104 may be based on a synchronization of the rendering of the media content 114 on the first electronic device 102.

In an embodiment, the second electronic device 104 may be at least one of the mobile device or the media playback device.

In an embodiment, the circuitry 202 may be further configured to receive identification information associated with the second electronic device 104. The circuitry 202 may be further configured to transmit the received identification information to the media server 106, wherein the control of the rendering of the audio content 114A on the second electronic device 104 may be further based on the transmitted identification information.

In an embodiment, the circuitry 202 may be further configured to determine a headphone signal (e.g., the headphone signal 406A) associated with the audio content 114A. The circuitry 202 may be further configured to transmit the determined headphone signal 406A to the second electronic device 104, based on the transmission of the content request, wherein the control of the rendering of the audio content 114A on the second electronic device 104 may be further based on the transmitted headphone signal 406A.

In an embodiment, the circuitry 202 may be further configured to determine the time information associated with the audio content 114A. The circuitry 202 may be further configured to synchronize the transmitted headphone signal 406A with the received media content 114 rendered on the first electronic device 102. Herein, the control of the rendering of the audio content 114A on the second electronic device 104 may be further based on the synchronization of the transmitted headphone signal 406A.

In an embodiment, the circuitry 202 may be further configured to control the execution of a hearing test of a user (e.g., the user 116) on the second electronic device 104. The circuitry 202 may be further configured to receive the hearing impairment information based on the execution of the hearing test. The circuitry 202 may be further configured to determine a hearing profile of the user 116 associated with the second electronic device 104 based on the received hearing impairment information, wherein the headphone signal 406A may be determined further based on the determined hearing profile.

In an embodiment, the circuitry 202 may be further configured to receive the hearing profile of the user 116 associated with the second electronic device 104, wherein the headphone signal 406A may be determined further based on the received hearing profile.

In an embodiment, the determined hearing profile may include at least one of a hearing capability of the user 116, an audio characteristic associated with the user 116, a preferred bass of the user 116, a preferred treble of the user 116, or a preferred amplitude of the user 116.

In an embodiment, the circuitry 202 may be further configured to apply an audio equalization on the audio content 114A based on the received hearing profile, wherein the headphone signal 406A may be determined further based on the application of the audio equalization.

In an embodiment, the circuitry 202 may be further configured to apply a dialogue enhancement on the audio content 114A based on the received hearing profile, wherein the headphone signal 406A may be determined further based on the application of the dialogue enhancement.

In an embodiment, the circuitry 202 may be further configured to receive a viewing profile of the user 116 associated with the second electronic device 104. The circuitry 202 may be further configured to determine an audio description associated with at least one portion of the received media content 114 based on the received viewing profile. The circuitry 202 may be further configured to insert the determined audio description in the determined headphone signal 406A.

In an embodiment, the circuitry 202 may be further configured to determine whether the received media content 114 may correspond to over-the-air (OTA) content. The circuitry 202 may be further configured to determine a broadcaster associated with the received media content 114 based on the determination that the received media content 114 corresponds to the OTA content. The circuitry 202 may be further configured to receive a uniform resource identifier (URI) associated with the media server 106 based on the determined broadcaster. The circuitry 202 may be further configured to transmit the received URI associated with the media server 106 to the second electronic device 104, based on the received user input, wherein the control of the rendering of the audio content 114A on the second electronic device 104 may be further based on the transmitted URI associated with the media server 106.

The present disclosure may also be positioned in a computer program product, which comprises all the features that enable the implementation of the methods described herein, and which when loaded in a computer system is able to carry out these methods. Computer program, in the present context, means any expression, in any language, code or notation, of a set of instructions intended to cause a system with information processing capability to perform a particular function either directly, or after either or both of the following: a) conversion to another language, code or notation; b) reproduction in a different material form.

While the present disclosure is described with reference to certain embodiments, it will be understood by those skilled in the art that various changes may be made, and equivalents may be substituted without departure from the scope of the present disclosure. In addition, many modifications may be made to adapt a particular situation or material to the teachings of the present disclosure without departure from its scope. Therefore, it is intended that the present disclosure is not limited to the embodiment disclosed, but that the present disclosure will include all embodiments that fall within the scope of the appended claims.

What is claimed is:

1. A first electronic device, comprising:
circuitry configured to:
receive media content including audio content;
receive a user input indicative of a request to render the audio content on a second electronic device associated with the first electronic device, wherein
a content request is transmitted to a media server including the audio content, based on the received user input;
render the received media content on the first electronic device; and
control the second electronic device to render the audio content, based on the transmitted content request, wherein
the rendering of the audio content on the second electronic device is based on a synchronization of the rendering of the received media content on the first electronic device.

2. The first electronic device according to claim 1, wherein the second electronic device is at least one of a mobile device or a media playback device.

3. The first electronic device according to claim 1, wherein the circuitry is further configured to:
receive identification information associated with the second electronic device; and
transmit the received identification information to the media server, wherein
the control of the rendering of the audio content on the second electronic device is further based on the transmitted identification information.

4. The first electronic device according to claim 1, wherein the circuitry is further configured to:
determine a headphone signal associated with the audio content; and
transmit the determined headphone signal to the second electronic device, based on the transmission of the content request, wherein
the control of the rendering of the audio content on the second electronic device is further based on the transmitted headphone signal.

5. The first electronic device according to claim 4, wherein the circuitry is further configured to:
determine time information associated with the audio content; and
synchronize the transmitted headphone signal with the received media content rendered on the first electronic device, wherein
the control of the rendering of the audio content on the second electronic device is further based on the synchronization of the transmitted headphone signal.

6. The first electronic device according to claim 4, wherein the circuitry is further configured to:
control an execution of a hearing test of a user on the second electronic device;
receive hearing impairment information based on the execution of the hearing test; and
determine a hearing profile of the user associated with the second electronic device based on the received hearing impairment information, wherein
the headphone signal is determined further based on the determined hearing profile.

7. The first electronic device according to claim 4, wherein the circuitry is further configured to:
receive a hearing profile of a user associated with the second electronic device, wherein
the headphone signal is determined further based on the received hearing profile.

8. The first electronic device according to claim 7, wherein the determined hearing profile includes at least one of a hearing capability of the user, an audio characteristic associated with the user, a preferred bass of the user, a preferred treble of the user, or a preferred amplitude of the user.

9. The first electronic device according to claim 7, wherein the circuitry is further configured to:
apply an audio equalization on the audio content based on the received hearing profile, wherein
the headphone signal is determined further based on the application of the audio equalization.

10. The first electronic device according to claim 7, wherein the circuitry is further configured to:
apply a dialogue enhancement on the audio content based on the received hearing profile, wherein
the headphone signal is determined further based on the application of the dialogue enhancement.

11. The first electronic device according to claim 4, wherein the circuitry is further configured to:
receive a viewing profile of a user associated with the second electronic device;
determine an audio description associated with at least one portion of the received media content based on the received viewing profile; and
insert the determined audio description in the determined headphone signal.

12. The first electronic device according to claim 1, wherein the circuitry is further configured to:
determine whether the received media content corresponds to over-the-air (OTA) content;
determine a broadcaster associated with the received media content based on the determination that the received media content corresponds to the OTA content;
receive a uniform resource identifier (URI) associated with the media server based on the determined broadcaster; and
transmit the received URI associated with the media server to the second electronic device, based on the received user input, wherein
the control of the rendering of the audio content on the second electronic device is further based on the transmitted URI associated with the media server.

13. A second electronic device, comprising:
circuitry configured to:
receive a user input indicative of a request to render audio content associated with media content on the second electronic device associated with a first electronic device, wherein
a content request is transmitted to a media server including the audio content, based on the received user input;
control the first electronic device to render the media content on the first electronic device; and
render the audio content, based on the transmitted content request, wherein
the rendering of the audio content on the second electronic device is based on a synchronization of the rendering of the media content on the first electronic device.

14. A method, comprising:
in a first electronic device:
receiving media content including audio content and video content;
receiving a user input indicative of a request to render the audio content on a second electronic device associated with the first electronic device, wherein
a content request is transmitted to a media server including the audio content, based on the received user input;
rendering the received media content on the first electronic device; and
controlling the second electronic device to render the audio content, based on the transmitted content request, wherein
the rendering of the audio content on the second electronic device is based on a synchronization of the rendering of the media content on the first electronic device.

15. The method according to claim 14, wherein the second electronic device is at least one of a mobile device or a media playback device.

16. The method according to claim 14, further comprising:
receiving identification information associated with the second electronic device; and
transmitting the received identification information to the media server, wherein
the control of the rendering of the audio content on the second electronic device is further based on the transmitted identification information.

17. The method according to claim 14, further comprising:
determining a headphone signal associated with the audio content; and
transmitting the determined headphone signal to the second electronic device, based on the transmission of the content request, wherein
the control of the rendering of the audio content on the second electronic device is further based on the transmitted headphone signal.

18. The method according to claim 17, further comprising:
determining time information associated with the audio content; and
synchronizing the transmitted headphone signal with the received media content rendered on the first electronic device, wherein
the control of the rendering of the audio content on the second electronic device is further based on the synchronization of the transmitted headphone signal.

19. The method according to claim 17, further comprising:
receiving a hearing profile of a user associated with the second electronic device, wherein
the headphone signal is determined further based on the received hearing profile.

20. The method according to claim 17, further comprising:
controlling an execution of a hearing test of a user on the second electronic device;
receiving hearing impairment information based on the execution of the hearing test; and
determining a hearing profile of the user associated with the second electronic device based on the received hearing impairment information, wherein
the headphone signal is determined further based on the determined hearing profile.
